(19) United States
(12) Patent Application Publication
(10) Pub. No.: US 2025/0032401 A1
(43) Pub. Date: Jan. 30, 2025

(54) COMPOSITION FOR IMPROVING SKIN CONDITION

(71) Applicant: CJ CHEILJEDANG CORPORATION, Seoul (KR)

(72) Inventors: Hee Yoon AHN, Suwon-si (KR); Hyun Sun YUN, Seoul (KR); Ji Young OH, Yongin-si (KR); Yun Hi CHO, Seoul (KR); Ji Hye SHIN, Seongnam-si (KR)

(73) Assignee: CJ CHEILJEDANG CORPORATION, Seoul (KR)

(21) Appl. No.: 18/905,553

(22) Filed: Oct. 3, 2024

Related U.S. Application Data
(62) Division of application No. 17/630,963, filed on Jan. 28, 2022.

(51) Int. Cl. A61K 8/99; A61K 8/60; A61Q 17/00; A61Q 19/00

(52) U.S. Cl. CPC A61K 8/99; A61K 8/60; A61Q 17/00; A61Q 19/00

(57) ABSTRACT

The present application relates to a composition which is for improving skin conditions and contains Lactobacillus plantarum CJLP55, the composition according to the present application suppress sebum, reduces oil or harmful bacteria in skin, and has a moisturizing effect, and thus the composition has an excellent effect on improving skin conditions and can be used for preventing or improving skin diseases such as acne and dermatitis.

### TECHNICAL FIELD

The present application relates to a composition which is for improving skin conditions and contains Lactobacillus plantarum CJLP55 or a culture solution thereof.

### BACKGROUND ART

In order to improve skin health and make skin beautiful, in recent years, interest in inner beauty through the intake of various materials and products has been increasing, in addition to the existing perception of focusing only on so-called outer beauty using cosmetics or the like applied to a skin surface. Skin problems, such as acne and dermatitis, which occur on a skin surface are known to be caused by increased sebum secretion, follicular hyperkeratinization, the formation of colonies of harmful skin bacteria, the occurrence of inflammation, and the like, and in connection with solving the root cause of such skin problems, research on the “gut-skin axis”, which is the theory about the correlation between the gastrointestinal system and the skin, has also been conducted.

Meanwhile, there have been attempts and research to use lactic acid bacteria, which are found in various fermented foods and known to be beneficial microorganisms for health, for the purpose of improving skin health. Moreover, there have been research results showing that antibacterial activity against harmful bacteria living in the skin is exhibited and the effect of improving wrinkles and aging of the skin is exhibited, when a culture solution of lactic acid bacteria or a composition containing the same was applied through methods such as application to the skin, and used as a cosmetic material. However, the effect on skin conditions when lactic acid bacteria are taken without being applied directly to the skin is unknown, and no research has been conducted on the effect.

Therefore, the inventors of the present application have discovered new effects and uses of a lactic acid bacteria strain, which can function as probiotics and are thus beneficial to health, have the efficacy of improving skin conditions, and can further significantly inhibit the growth and development of harmful skin bacteria compared to other strains, and thus have completed the invention of the present application.

### DISCLOSURE OF THE INVENTION

### Technical Problem

An object of the present application is to provide a composition which is for improving skin conditions, suppresses the occurrence and progression of skin problems such as acne, and can maintain moisturizing while reducing oil in skin.

### Technical Solution

In order to achieve the object, the present application provides a composition which is for improving skin conditions and contains Lactobacillus plantarum CJLP55 or a culture solution thereof.

### Advantageous Effects

The composition for improving skin conditions according to the present application has an advantage of improving skin conditions and suppressing the occurrence and progression of skin problems, by improving the severity of acne on the basis of a growth and development inhibitory effect on harmful skin bacteria such as the genus Propionibacterium, reducing oil and sebum in skin, maintaining the amount of moisture, and improving the acidity of the skin.

However, the effects of the present application are not limited to the aforementioned effects, and other effects, which are not mentioned, will be clearly understood by a person with ordinary skill in the art from the following description.

### BRIEF DESCRIPTION OF THE DRAWINGS

FIG. 1a and FIG. 1b are graphs showing a comparison of the numbers of subjects whose the oil is improved when oil is measured after subjects in a lactic acid bacteria (Lactobacillus plantarum CJLP55) intake group and a placebo group are asked to take each food for 12 weeks in a human application test, FIG. 1a is a graph showing a comparison of the numbers of subjects whose the oil is improved by 30% or more and FIG. 1b is a graph showing a comparison of the numbers of subjects whose the oil is improved by 50% or more, and in each graph, the left side represents an ITT analysis result and the right side represents a PP analysis result; and

FIG. 2 is graphs showing, as a ratio of each species of microorganisms belonging to the genus Propionibacterium, ITT (A of FIG. 2) and PP (B of FIG. 2) analysis results obtained by analyzing the base sequence of skin flora in the human application test, and in both of the two graphs, P. acnes is 94%, P. unassigned is 5%, and P. granulosum is 1%.

### MODE FOR CARRYING OUT THE INVENTION

Hereinafter, the present application will be described in detail.

The present application provides a composition which is for improving skin conditions and contains Lactobacillus plantarum CJLP55 or a culture solution thereof.

The term “culture solution” in the present application refers to a culture solution itself obtained by culturing a strain, or a culture supernatant obtained by removing the strain from the same, and a filtrate, concentrate, or dried substance thereof, and may be used interchangeably with a “culture supernatant”, a “conditional culture solution”, or a “conditioned medium”.

The term “improvement” in the present application refers to all actions including recovery, suppression, or delay of symptoms, and may be used interchangeably with prevention or treatment.

The prevention may be any action of suppressing the disease or delaying the onset of the disease by administering a composition to a subject, and the treatment may be any action of recovering the symptoms of the disease, which is already affected, by administering a composition to a subject.

The term “improving skin conditions” in the present application refers to any action of improving skin conditions, regardless of a cosmetic aspect or a medical aspect, and may be used interchangeably with improving skin health or improving hypersensitive skin conditions.

The Lactobacillus plantarum CJLP55 is a type of lactic acid bacteria, is a gram-positive bacterium, and has a characteristic of being able to grow under both aerobic/anaerobic conditions. Specifically, the Lactobacillus plantarum CJLP55 may be a microorganism deposited with the accession number KCTC 11401BP (depository institution: Korean Collection for Type Culture, deposit date: Oct. 16, 2008), and may be separated from fermented foods such as kimchi, fermented vegetables, soybean paste, soy sauce, cheonggukjang, and salted seafood. The Lactobacillus plantarum CJLP55 has excellent acid resistance to gastric acid, bile acid resistance to bile acid, and adhesion to intestinal epithelial cells, and thus can have a beneficial effect on the intestinal flora in the gastrointestinal tract of an animal including a human, and can be used as a probiotic.

The culture solution is obtained by culturing the Lactobacillus plantarum CJLP55, may be a culture solution itself containing the lactic acid bacteria cells, or a culture supernatant obtained by removing the lactic acid bacteria cells from the same, and may be a filtrate, concentrate, or dried substance thereof. The culture solution from which the lactic acid bacteria cells are removed contains components produced and secreted by the Lactobacillus plantarum CJLP55, and thus may have skin condition-improving activity.

The filtrate is obtained by removing floating solid particles from the culture solution of the Lactobacillus plantarum CJLP55 and collecting only a water-soluble supernatant excluding a precipitate, and a filter such as cotton or nylon, for example, a filter of 0.2 μm to 5 μm may be used to filter particles, or a cryofiltration method, a centrifugal separation method, or the like may be used, but the method is not limited thereto.

The concentrate is obtained by increasing the solid content concentration of the culture solution, and may be a concentrate of a culture solution containing the lactic acid bacteria cells, or a concentrate of a culture supernatant from which the lactic acid bacteria cells are removed. The concentrate may be a substance concentrated by vacuum concentration, plate-shaped concentration, thin film concentration, or the like, but is not limited thereto, and the concentration can be performed, for example, at a temperature of 40° C. to 60° C. using a well-known concentrator. The content of the culture solution contained in the composition according to the present application can be appropriately adjusted according to the concentration of the concentrate.

The dried substance includes a substance dried through methods such as freeze drying, vacuum drying, hot air drying, spray drying, reduced-pressure drying, foam drying, high-frequency drying, and infrared drying, but is not limited thereto. For example, when a freeze-dried substance obtained by freeze-drying a culture solution containing Lactobacillus plantarum CJLP55 cells is used, there is an advantageous advantage in oral intake, forming into a dosage form, packaging, or storage of a composition containing the freeze-dried substance, and since the strain can be preserved for a long period of time, there is an advantage that the strain, which has been freeze-dried in a subject to which the composition according to the present application is administered, particularly in the intestine, exhibits normal physiological activity again, and can perform growth and metabolism.

The Lactobacillus plantarum CJLP55 or a culture solution thereof may be contained in an amount of 3 wt % to 15 wt % in the composition according to the present application. Specifically, the Lactobacillus plantarum CJLP55 or a culture solution thereof may be contained in an amount in a range including one lower limit selected from 3 wt %, 3.5 wt %, 4 wt %, 4.5 wt %, 5 wt %, 5.5 wt %, 6 wt %, 6.5 wt %, and 7 wt % and/or one upper limit selected from 15 wt %, 14 wt %, 13 wt %, 12 wt %, 11 wt %, 10.5 wt %, 10 wt %, 9.5 wt %, and 9 wt %. As one example, the content thereof may be 3 to 15 wt %, 3.5 to 14 wt %, 4 to 13 wt %, 4.5 to 12 wt %, 5 to 11 wt %, 5.5 to 10.5 wt %, 6 to 10 wt %, 6.5 to 9.5 wt %, or 7 to 9 wt %. In a case where the content of the Lactobacillus plantarum CJLP55 or a culture solution thereof is within the above range, the intake thereof enables functions such as proliferation of beneficial lactic acid bacteria in the intestine, suppression of harmful bacteria, and a smooth bowel movement, and the effect of improving skin conditions may be sufficiently improved, and there may be no sense of rejection in terms of taste and aroma when trying to take the composition according to the present application.

The composition may further contain maltodextrin and glucose. The maltodextrin and the glucose may be contained as excipients. The composition may contain, without limitation, any component, which can be commonly used as an excipient in the technical field of the present application, as long as the component does not inhibit the effect of improving skin conditions by the Lactobacillus plantarum CJLP55 or a culture solution thereof contained in the composition according to the present application. By incorporating the maltodextrin and the glucose in the composition according to the present application, an effect of suppressing the secretion of free fatty acids from epidermal cells and/or sebaceous glands of a person who takes the composition can be exhibited. The contents of the maltodextrin and the glucose may be set without limitation at a level which does not inhibit the effect of improving skin conditions by the Lactobacillus plantarum CJLP55 or a culture solution thereof, and for example, the maltodextrin may be contained in the same amount as the glucose in the composition according to the present application.

The composition according to the present application may be for oral administration. The “for oral administration” means that the composition according to the present application is used to be administered/taken through the oral cavity of a human or animal, and thus the composition according to the present application can be used in the form of a pharmaceutical product or food. When the composition is orally administered, the Lactobacillus plantarum CJLP55 contained in the composition can function as a probiotic in the gastrointestinal tract of a human or animal, and can also affect skin to exhibit an effect that can improve skin conditions.

The improving skin conditions may be one or more selected from the group consisting of sebum suppression, sebum component improvement, acne improvement, and dermatitis improvement. The composition according to the present application can suppress sebum by reducing the amount of sebum or by reducing the secretion thereof. Moreover, the sebum component can be improved by reducing the content of components, which may cause skin problems among the components in sebum, such as a triglyceride, cholesterol, a cholesterol ester, and a free fatty acid, or by increasing the content of components such as a ceramide, which may improve skin conditions, such as exhibiting an effect of maintaining skin moisturizing, among the components in sebum. The acne improvement may be a decrease in a nodule, a papule, a pustule, and a comedone, or a decrease in the severity of acne. Furthermore, the composition according to the present application can improve dermatitis, which can be caused by sebum, by suppressing the secretion of sebum to reduce the amount of sebum, and improving the components in sebum.

The composition may reduce one or more selected from the group consisting of a triglyceride, cholesterol, a cholesterol ester, and a free fatty acid in sebum, or increase a ceramide. The triglyceride may be secreted from a sebaceous gland, and the cholesterol, the cholesterol ester, and the free fatty acid may be secreted from both a sebaceous gland and epidermis. The ceramide may be a ceramide 1 to a ceramide 7, and specifically a ceramide 2, and as the ceramide 2 increases, an effect of improving skin moisturizing can be exhibited.

The composition may reduce oil in skin and increase moisture in skin. Specifically, the composition according to the present application may reduce the amount of oil (for example, a triglyceride, cholesterol, a cholesterol ester, a free fatty acid, and the like) on a skin surface, or reduce the amount of sebum. Moreover, the composition can maintain the amount of moisture by increasing the amount of moisture in skin or inhibiting a decrease in moisture, and thus can exhibit a skin moisturizing effect.

The composition may improve the acidity of skin. The improvement in the acidity refers to reducing the pH of skin or inhibiting an increase in the pH of skin. The acidity of skin can be used as a key indicator of skin health, and healthy skin generally maintains weak acidity of pH of 4 to pH of 6, whereas the pH of skin may increase when skin diseases such as atopic dermatitis and acne occur. Thus, reducing the pH can improve skin conditions. The acidity of skin can be determined by the total content of lactic acid, a free amino acid, a free fatty acid, and the like produced in epidermis, and the composition may increase the content of lactic acid in the skin.

The composition may have antibacterial activity against microorganisms belonging to the genus Propionibacterium (the genus Cutibacterium) or the genus Staphylococcus. The microorganisms belonging to the genus Propionibacterium or the genus Staphylococcus may cause skin diseases such as acne and/or dermatitis. The composition according to the present application may have antibacterial activity by inhibiting the growth of the microorganisms, and may reduce the number of microorganisms or inhibit an increase in the number of microorganisms. For example, the composition may have antibacterial activity against the microorganisms distributed on a skin surface, and thus may prevent, treat, and improve skin diseases caused by the microorganisms.

The microorganisms belonging to the genus Propionibacterium may be Propionibacterium acnes (or Cutibacterium acnes). The Propionibacterium acnes is a harmful skin bacterium which may cause acne or dermatitis, and can grow using fatty acids secreted from sebaceous glands. The composition according to the present application may have antibacterial activity by inhibiting the growth of the Propionibacterium acnes, and thus may prevent, treat, and improve skin diseases caused by the Propionibacterium acnes.

The composition according to the present application can be used as a food composition or a pharmaceutical composition, for the purpose of improving skin conditions.

When the composition according to the present application is used as a food composition, the types of the food include meat, sausage, bread, chocolate, candies, snacks, confectionery, pizza, ramen, other noodles, gums, a dairy product including ice creams, various soups, a beverage, tea, a health drink, an alcoholic beverage, a vitamin complex, a functional food, a health functional food, and the like, and include all foods in the usual sense, and for example, the food composition may be a composition in which the Lactobacillus plantarum CJLP55 or a culture solution thereof, which is an effective component contained in the composition, is not destroyed or does not lose functions during food processing, a cooking process, or the like.

The health functional food has antibacterial activity against harmful skin bacteria by reducing sebum or oil and maintaining moisturizing, and thus can prevent/improve acne and/or dermatitis, or improve skin conditions. A health functional food, which contains, as an effective component, Lactobacillus plantarum CJLP55 or a culture solution thereof having an effect of improving skin conditions, can be usefully used for preventing or improving acne and/or dermatitis, and improving skin conditions.

The health functional food is a food which has high medical and medical treatment effects and is processed to effectively exhibit a living body-modulating function in addition to nutrition supply, and can achieve useful effects for a health use, such as nutrient controlling or physiological actions on the structure and function of the human body. The health functional food may be produced by a method commonly used in the technical field of the present application, and may be produced by adding raw materials and components commonly added in the art. Moreover, the dosage form of the health functional food may be produced in various dosage forms without limitation as long as the dosage form is a dosage form recognized as a health functional food, and the health functional food has an advantage that side effects and the like, which may occur when taking the drug for a long time, are not caused by using a food as a raw material, unlike general drugs, and an advantage of excellent portability, and thus can be used simultaneously with or separately from a medicine for treatment, before the onset stage of the disease or after the onset of the disease in order to prevent or improve acne and/or dermatitis. The health functional food includes a health food having an active health maintenance or promotion effect compared to general foods and a health supplement food for health supplement purposes, and the terms health functional food, health food, and health supplement food may be interchangeably used in some cases.

In the health functional food, the effective component (Lactobacillus plantarum CJLP55 or a culture solution thereof) may be added to the food as it is or may be used together with other foods or food components, and may be appropriately used according to an ordinary method. The mixed amount of the effective component can be appropriately determined according to the purpose of usage (for preventing or improving acne and/or dermatitis, and for improving skin conditions). The effective component may be contained in various amounts in the health functional food as long as the effective component has effects of preventing or improving acne and/or dermatitis and improving skin conditions. However, in the case of long-term intake for the purpose of health and hygiene or for health control purposes, the amount may be equal to or less than the above range. The health functional food may contain other components as essential components without particular limitation, in addition to containing the effective component. For example, like ordinary beverages, various flavoring agents, natural carbohydrate, or the like may be contained as an additional component. Examples of the natural carbohydrate may include: common sugars, for example, a monosaccharide such as glucose and fructose, a disaccharide such as maltose and sucrose, and a polysaccharide such as dextrin and cyclodextrin; and sugar alcohols such as xylitol, sorbitol, and erythritol. As flavoring agents other than the aforementioned flavoring agents, a natural flavoring agent (thaumatin and a stevia extract (for example, rebaudioside A, glycyrrhizin, and the like)) and a synthetic flavoring agent (saccharin, aspartame, and the like) can be advantageously used. The proportion of the natural carbohydrate can be appropriately determined by the choice of a person with ordinary skill in the art.

The health functional food may contain various nutritional agents, a vitamin, a mineral (electrolyte), a flavor such as a synthetic flavor and a natural flavor, a coloring agent and a thickening agent (cheese, chocolate, or the like), pectic acid and a salt thereof, alginic acid and a salt thereof, an organic acid, a protective colloid thickener, a pH adjuster, a stabilizer, an antiseptic agent, glycerin, alcohol, a carbonation agent used in carbonated beverages, or the like. These components can be used independently or in combination, and the proportions of these additives can also be appropriately selected by a person with ordinary skill in the art.

When the composition according to the present application is used as a pharmaceutical composition, the pharmaceutical composition may be for preventing or improving acne and/or dermatitis.

The composition may be administered together with one or more effective components exhibiting the same or similar function. For the administration, one or more additionally acceptable carriers may be contained according to a method which can be easily performed by a person with ordinary knowledge in the technical field to which the present application belongs. The expression “acceptable” means that a substance to be applied (prescribed) does not have toxicity beyond adaptable toxicity without suppressing the activity of the effective component (Lactobacillus plantarum CJLP55 or a culture solution thereof). The “carriers” are defined as compounds that facilitate the addition of a compound into cells or tissues. The composition may be produced in a unit dose form by being formulated using a carrier and/or an excipient, or may be produced by being placed into a multi-dose container, and may additionally contain a dispersing agent or a stabilizer. Moreover, the effective component contained in the composition may be carried on a carrier such as a colloidal suspension, a powder, a saline solution, a lipid, a liposome, microspheres, or nano-spherical particles. The effective component may form a complex with or be associated with a carrying mean, and may be carried into a living body using a carrying system, which is well known in the technical field to which the present application belongs, such as a lipid, a liposome, fine particles, gold, nanoparticles, a polymer, a condensation reaction agent, polysaccharides, a polyamino acid, a dendrimer, a saponin, an adsorption enhancing substance, or a fatty acid. In addition, the carrier may contain lactose, dextrose, sucrose, sorbitol, mannitol, starch, acacia, rubber, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil, and the like, which are commonly used during formulation, but is not limited thereto. Furthermore, in addition to the aforementioned components, a lubricant, a wetting agent, a sweetening agent, a flavoring agent, an emulsifying agent, a suspending agent, a preservative, and the like may be further contained. As the carrier, a saline solution, sterile water, Ringer's solution, a buffered saline solution, a dextrose solution, a maltodextrin solution, glycerol, ethanol, and a mixture of one or more of these components may be used, and other ordinary additives such as an antioxidant, a buffer solution, and a bacteriostatic agent may be added, if necessary.

When the composition according to the present application is pharmaceutically used, the composition can be administered in various oral and parenteral dosage forms at the time of actual clinical administration, and in the case of being formulated, the composition is prepared using a diluent or an excipient, such as a filler, an extender, a bonding agent, a wetting agent, a disintegrating agent, and a surfactant, which are commonly used. A solid formulation for oral administration includes a tablet, a pill, a powder, a granule, a capsule, and the like, and such a solid formulation is prepared by mixing at least one excipient such as starch, calcium carbonate, sucrose, lactose, gelatin, or the like with an herbal medicine extract or a fermented herbal medicine. Moreover, in addition to the simple excipients, lubricants such as magnesium stearate and talc are also used. The powder may be produced by simply mixing the effective component according to the present application with a suitable pharmaceutically acceptable carrier such as lactose, starch, and microcrystalline cellulose. The granule may be produced through a wet granulation method using a solvent such as water, ethanol, and isopropanol, or a dry granulation method using a compressive force, after mixing the effective component according to the present application, a suitable pharmaceutically acceptable carrier, and a suitable pharmaceutically acceptable bonding agent such as polyvinyl pyrrolidone and hydroxypropyl cellulose. Furthermore, the tablet may be produced by mixing the aforementioned granule with a suitable pharmaceutically acceptable lubricating agent such as magnesium stearate, and then tableting the mixture using a tablet machine. A liquid formulation for oral administration includes a suspending agent, an oral liquid, an emulsion, a syrup, and the like, and various excipients such as a wetting agent, a sweetening agent, a fragrance, and a preservative, in addition to water and liquid paraffin, which are commonly used simple diluents, may be contained. A formulation for parenteral administration includes a sterilized aqueous solution, a non-aqueous solvent, a suspending agent, an emulsion, a freeze-dried formulation, and a suppository. As the non-aqueous solvent and the suspending solvent, propylene glycol, polyethylene glycol, vegetable oil such as olive oil, an injectable ester such as ethyl oleate, and the like can be used. As a base for the suppository, Witepsol, Macrogol, Tween 61, cacao butter, laurin butter, glycerol, gelatin, and the like can be used.

When the composition according to the present application is pharmaceutically used, the effective component (Lactobacillus plantarum CJLP55 or a culture solution thereof) may be administered as an oral agent, an injection (for example, an intramuscular injection, an intraperitoneal injection, an injection, intravenous an infusion, a subcutaneous injection, and an implant), an inhalant, an intranasal administration agent, a vaginal agent, a rectal administration agent, a sublingual agent, a transdermal agent, a topical agent, and the like, depending on the disease to be prevented, improved, or treated and the condition of an individual, but is not limited thereto. According to an administration route, the composition may be formulated into a suitable administration unit dosage form including a pharmaceutically acceptable carrier, additive, or vehicle, which is commonly used and non-toxic. The range of the amount of the Lactobacillus plantarum CJLP55, which is an effective component, at the time of the administration varies depending on the weight, age, sex, health status, and diet of a patient, an administration time, an administration method, an excretion rate, a target site, the severity of a disease, and the like. The effective component in the composition may be contained in a concentration of 30 μM or more, for example, 32 μM or more, 35 μM or more, 37 μM or more, or 40 μM or more. Moreover, the effective component may be contained in an amount in a range including one lower limit selected from 0.05 mg, 0.1 mg, 0.15 mg, 0.2 mg, 0.3 mg, 0.5 mg, 1 mg, 2 mg, 3 mg, 5 mg, 10 mg, 20 mg, 30 mg, 50 mg, and 100 mg and/or one upper limit selected from 500 mg, 450 mg, 400 mg, 350 mg, 320 mg, 300 mg, 280 mg, 250 mg, 200 mg, 150 mg, and 100 mg. As one example, the effective component may be contained in an amount of 0.05 to 500 mg, 0.05 to 450 mg, 0.05 to 400 mg, 0.05 to 350 mg, 0.05 to 300 mg, 0.05 to 250 mg, 0.1 to 500 mg, 0.1 to 450 mg, 0.1 to 400 mg, 0.1 to 350 mg, 0.1 to 300 mg, 0.1 to 250 mg, 0.1 to 200 mg, 0.2 to 500 mg, 0.2 to 400 mg, 0.2 to 300 mg, 0.5 to 300 mg, 1 to 300 mg, 5 to 300 mg, or 10 to 300 mg.

In addition, when the composition is pharmaceutically used, the in a composition may be administered pharmaceutically effective amount. The “pharmaceutically effective amount” in the present application refers to an amount sufficient to treat a disease with a reasonable benefit/risk ratio applicable to medical treatment, and an effective dose level can be determined according to factors including the type and severity of the disease of a patient, the activity of a drug, sensitivity to a drug, an administration time, an administration route, an excretion rate, a treatment period, and a concurrently used drug, and other factors well known in the medical field. The effective dose is generally 0.01 mg to 5,000 mg per 1 kg of the body weight of an administered individual per day, and the administration may be performed once or several times a day at regular time intervals according to the judgment of a doctor or a pharmacist, but is not limited thereto. The composition may be administered as an individual therapeutic agent or may be administered in combination with a therapeutic agent for diseases caused by other contaminants or a therapeutic agent for improving skin aging, may be administered simultaneously, separately, or sequentially with a therapeutic agent in the related and may be administered singly or in multiples. It is important to administer an amount capable of achieving the maximum effect in the minimum amount without causing side effects, in consideration of all of the aforementioned factors, and the amount can be easily determined by a person with ordinary skill in the art. Specifically, the effective amount of the composition may vary depending on the age, sex, condition, weight of a patient, the absorption degree of an active component in the body, an inactivation rate, an excretion speed, a disease type, and drugs used in combination, may be increased or decreased depending on an administration route, the severity of obesity, a sex, a weight, an age, and the like, and may vary depending on the severity of the condition being treated and the like. If necessary, the total daily dosage may be dividedly administered several times during a day for convenience. As one example, the daily dosage may be about 0.0001 mg/kg to about 10 g/kg per day, and for example, an amount of about 0.001 mg/kg to about 1 g/kg may be administered once a day. Moreover, the administration period may be one day to two months, but the administration may be performed without limitation until an effect of preventing or treating a disease is achieved. Furthermore, the administration may be dividedly performed several times a day, for example, twice or three times a day, at regular time intervals according to the judgment of a doctor or pharmacist.

Another aspect of the present application provides a method for improving skin conditions, the method including a step of administering, to a subject, a composition which contains Lactobacillus plantarum CJLP55 or a culture solution thereof.

The subject may include any subject without limitation, but may be an animal including a human or an animal not including a human, for example. When the skin health condition of a subject to which the composition is to be administered is a negative condition compared to a normal individual, or is a condition with a skin disease, for example, when the skin health condition is a condition such as increased sebum, acne, or dermatitis, the skin condition can be treated or improved by administering the composition. Moreover, when the skin health condition of a subject to which the composition is to be administered is normal, it is possible to prevent or inhibit the onset of a skin disease and the skin conditions of the subject from reaching a negative condition, by administering the composition.

The administration may be oral administration of the composition to a subject. The description for the oral administration is the same as described above.

A step of preparing a composition, which contains Lactobacillus plantarum CJLP55 or a culture solution thereof, may be further included before the step of administering the composition to the subject. The step of preparing a composition may be using the Lactobacillus plantarum CJLP55 or a culture solution thereof as an effective component, and further mixing other components such as a carrier and an excipient. The descriptions for the Lactobacillus plantarum CJLP55, the culture solution thereof, the skin condition improvement, the carrier, the excipient, the other components, the administration method, the dosage, and the like are the same as described above.

Hereinafter, the present application will be described in detail with reference to experimental examples.

However, the following experimental examples are to specifically illustrate the present application, and the contents of the present application are not limited by the following examples.

### Experimental Example 1

### Checking of Growth and Development Inhibitory Effect of Lactobacillus Plantarum CJLP55 Against Causative Bacteria (P. Acnes) of Acne

In order to check a growth and development inhibitory effect of a Lactobacillus plantarum CJLP55 strain against Propionibacterium acnes (or Cutibacterium acnes, and hereinafter, ‘P. acnes’) known as a causative bacterium causing skin problems such as acne, P. acnes was inoculated together with various types of lactic acid bacteria in a culture medium, and whether or not the proliferation of the P. acnes was suppressed was checked in vitro.

As the Lactobacillus plantarum CJLP55 strain, a strain disclosed in Korean Registered Patent Publication No. 10-1255050 B1 and deposited with KCTC 11401BP on Oct. 16, 2008 in Korean Collection for Type Culture was used.

As the lactic acid bacteria strain, the reference strain KCTC 3108 of Lactobacillus plantarum was used, and Lactobacillus rueteri KCTC 3594 and the Lactobacillus plantarum CJLP55 strain according to the present application, which are well known (Mi-Sun Kang et al., 2012, The Journal of Microbiology, 50 (1), pp. 137 to 142) to have activity to suppress the growth and development of the P. acnes were used. The P. acnes (accession number: KCTC 3314, culture medium: reinforced clostridial medium, RCM medium, and culture temperature: 37° C.) as a harmful bacterium causing acne was sealed using a gas pack system (AnaeroPack, MITSUBISHI GAS CHEMICAL COMPANY, INC.) in an anaerobic culture tank, and cultured under anaerobic conditions.

The Lactobacillus plantarum CJLP55 strain according to the present invention, the Lactobacillus plantarum KCTC 3108 as a reference strain, and the Lactobacillus rueteri KCTC 3594 strain were dripped in an MRS medium (Difco 0881) by 1.0×10⁷ CFU/ml, and cultured for 24 hours, then the P. acnes strain was inoculated in an RCM medium containing 0.8% of agar by 1.0×10⁷ CFU/ml, and the media were overlaid with each other. After additional culturing for 24 hours, the diameter of an inhibition zone (clear zone), which appeared by suppressing the growth of the P. acnes strain around the clusters of each experimental strain and a control strain, was measured.

As a result, as shown in Table 1 below, inhibition zones were not formed at all in the cluster of the reference strain of the Lactobacillus rueteri, but inhibition zones were formed in the clusters of the reference strain (KCTC 3108) of the Lactobacillus plantarum and the Lactobacillus plantarum CJLP55 strain according to the present invention. Here, it was found that the diameter of the inhibition zone formed around the cluster of the Lactobacillus plantarum CJLP55 strain was larger compared to the case of the cluster of the reference strain. Since the diameter size of the inhibition zone is larger when the antibacterial activity exhibited by the strain of the corresponding cluster is excellent, it was confirmed that the Lactobacillus plantarum CJLP55 strain according to the present invention exhibited antibacterial activity to suppress the growth of the P. acnes, and had superior antibacterial activity against the P. acnes compared to the general reference strain of Lactobacillus plantarum.

TABLE 1

| | Diameter (mm) of |
|---|---|
| | inhibition zone |
| Reference strain (KCTC 3594) | − |
| of Lactobacillus rueteri | |
| Reference strain (KCTC 3108) | ++ |
| of Lactobacillus plantarum | |
| Lactobacillus plantarum | +++ |
| CJLP55 | |

(−: no inhibition zone, +: smaller than 10.0 mm, ++: 10.0 to 20.0 mm, and +++: larger than 20.0 mm)

From the above results, it was newly confirmed that the Lactobacillus plantarum CJLP55 strain according to the present invention exhibits superior activity to suppress the growth of the P. acnes compared to other lactic acid bacteria strains in the related art known to be able to suppress the activity of harmful bacteria causing acne to some extent, and has superior activity to suppress the growth of harmful bacteria causing acne, in particular, compared to KCTC 3108 as the reference strain of Lactobacillus plantarum. Therefore, the Lactobacillus plantarum CJLP55 according to the present invention or a culture solution containing the same can be used as a component for improving acne, which may be caused by the P. acnes strain, and the like, and thus can be usefully used as an effective component of a composition for improving skin conditions.

### Experimental Example 2

### Checking of Effect of Reducing Sebum and Improving Skin Problems by Intake of Lactobacillus Plantarum CJLP55

A human application test was performed in order to check an effect of reducing sebum and improving skin problems in the human body when taking Lactobacillus plantarum CJLP55, which had been confirmed to have an excellent effect of suppressing the growth and development of P. acnes as a causative bacterium causing acne. The human application test was conducted on 90 adults with the approval (approval number: KHSIRB 18-016) of the Ethics Committee of Kyung Hee University.

### [2-1] Selection of Research Subject and Assignment of Experimental Group

A sebum standard for each site (SM315 sebum meter, Courage+Khazaka electronic GmbH, Germany) according to Table 2 below, and an acne severity evaluation system (Investigator's Global Assessment (IGA), provided by National Institute of Food and Drug Safety Evaluation) according to Table 3 below were applied for healthy men and women aged 18 to 39 with acne, and the department of dermatology of Wonju Severance Christian Hospital recruited and selected subjects whose the sebum secretion level in a forehead and a T-zone area was “oily” and the acne severity was rated 2 to 3. Moreover, while maintaining double-blindness, the subjects were classified into a placebo group and a lactic acid bacteria (Lactobacillus plantarum CJLP55) intake group to perform a test.

Statistical processing was performed separately for the results (ITT, intent to treat) of all subjects in the human application test, and the results (PP, per protocol) of subjects, excluding people whose the oil level was suitable as a subject at the time of selection but was measured to be 85 or higher at the 0^{th} week, or subjects who took antibiotics and was received dermatological procedures during the intake period of 12 weeks, among the subjects.

TABLE 2

| | Forehead, T-zone, and scalp |
|---|---|
| Less sebum | <70 |
| Normal | 70 to 150 |
| Oily | >150 |

TABLE 3

| Rating | Characteristics |
|---|---|
| 0 | No lesion |
| 1 | Almost no lesions (nodule: none, papule/pustule: |
| | one or less, and comedone: few) |
| 2 | Mild (nodule: none, papule/pustule: five or less, |
| | and comedone: a few) |
| 3 | Moderate (nodule: one or less, papule/pustule: six |
| | or more, and comedone: many) |
| 4 | Severe (nodule: five or less, papule/pustule: many, |
| | and comedone: many) |

### [2-2] Production of Intake Food and Intake Method

A placebo (control food) to be taken by the placebo group was a product which did not contain Lactobacillus plantarum CJLP55, and was produced to contain 50 wt % of maltodextrin and 50 wt % of powdered glucose. A food to be taken by the lactic acid bacteria intake group was produced to contain 9 wt % of Lactobacillus plantarum CJLP55, 45.5 wt % of maltodextrin, and 45.5 wt % of powdered glucose. The respective foods produced as described above have the same shape, color, and scent, and regarding the intake amounts and the intake methods, the foods were taken once a day regardless of a meal for a total of 12 weeks by putting one bag (2 g) once in the morning or in the evening as it was into the mouth, or by taking a mixture with 20 ml to 40 ml of water or milk, and when taking other drugs, the subjects were asked to take the other drugs at intervals of 2 hours.

In the case of the lactic acid bacteria intake group, the Lactobacillus plantarum CJLP55 was taken so that the intake amount was 10¹⁰ CFU/day corresponding to the minimum amount in which the probiotics reached the intestine to exert a beneficial function.

### [2-3] Statistical Analysis Method

Descriptive statistics of all data obtained through the human application test were performed using an SPSS 23.0 program (IBM, USA). Regarding the data of all subjects, the student's paired t-test was performed for the measurement results before and after (0^{th} and 6^{th} weeks or 0^{th} and 12^{th} weeks) the intake, and the student's unpaired t-test was performed for a significance between groups during the intake period of 12 weeks. The significance was verified at P-value<0.05.

### [2-4] Checking of Effect of Improving Oil, Moisture, and pH by Intake of Lactic Acid Bacteria

The placebo group and the lactic acid bacteria intake group were first asked to take the food, which was produced according to Experimental Example 2-2, according to the aforementioned intake method for 12 weeks, and then changes in the oil, moisture, and pH of skin were checked. At the 0^{th}, 6^{th}, and 12^{th} weeks, the subjects were washed their faces and asked to stay for 30 minutes or longer in a space where a temperature of 18° C. to 26° C. and a humidity of 26% to 53% were maintained, and then the oil, moisture, and pH in the forehead and the T-zone area were measured through a sebum meter (SM815, Courage+Khazaka electronic GmbH, Germany), a corneum meter (CM825), and a pH meter (PH905) using a probe. The levels of the moisture, oil, and acidity for each week of each subject are the averages of values obtained through measurements performed repeatedly five times.

Among the measurement results of the oil, the moisture, and the pH, the measurement results for 42 subjects in the placebo group and 42 subjects in the lactic acid bacteria intake group, which correspond to the ITT analysis results, are shown in Table 4 below, and the measurement results for 39 subjects in the placebo group and 39 subjects in the lactic acid bacteria intake group, which correspond to the PP analysis results, are shown in Table 5 below.

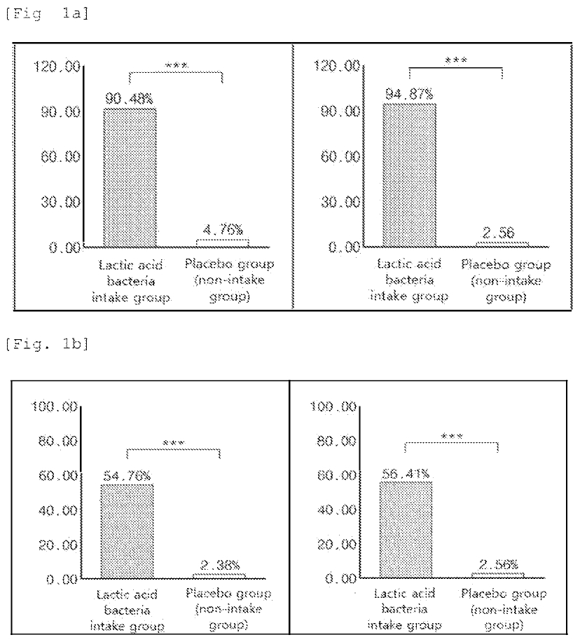

As the result of the ITT analysis on the change in the oil, the ranges of individual oil levels in the placebo group were measured to be gradually increased, whereas the ranges of individual oil levels in the lactic acid bacteria intake group were measured to be gradually decreased with increasing the intake period. The oil contents of the lactic acid bacteria intake group at the 6^{th} week and the 12^{th} week were found to be significantly decreased compared to the 0^{th} week, and a significance between groups also appeared compared to the placebo group. Also in the case of the result of the PP analysis on the change in the oil, unlike the placebo group, the range of the oil level was decreased as the intake period elapsed, the oil contents at the 6^{th} week and the 12^{th} week were significantly decreased compared to the 0^{th} week. When compared to the placebo group, it was also found that a significance between groups appeared. From the ITT and PP analysis results, it was confirmed that the effect of reducing oil and sebum by the intake of the Lactobacillus plantarum CJLP55 was significant from the 6^{th} week, and the effect was remarkable at the 12^{th} week. In particular, when compared to the 0^{th} week, the number of subjects whose the oil is improved by 30% or 50% or more was statistically significantly higher in both the ITT and PP analysis results (FIG. 1a and FIG. 1b).

In the case of the ITT and PP analysis on the change in the epidermal moisture, which is an indicator of skin moisturizing, it was confirmed that moisturizing was improved in both the placebo group and the lactic acid bacteria intake group up to the 6^{th} week and a significance between groups did not appear, but moisturizing was more significantly increased in lactic acid bacteria intake group than in the placebo group from the 12^{th} week and a significance between groups appeared.

Also in the case of the results of the ITT and PP analysis on the change in the skin acidity measured through a pH, the acidity improvement effect was significant up to the 6^{th} week and there was no significance between groups, but the acidity was more significantly improved in the lactic acid bacteria intake group with a decrease in the pH at the 12^{th} week and a significance between groups appeared.

TABLE 4

| | | | | Lactic acid | P-value |
|---|---|---|---|---|---|
| | | | | bacteria | (significance |
| | | | Placebo group | intake group | between |
| ITT | | | (n = 42) | (n = 42) | groups) |
| Sebum | 6^{th} | Change amount (%) | −2.11 ± 1.99 | −22.74 ± 2.25 | 0.000 |
| (μg/cm²) | week | compared to 0^{th} week | | | |
| | | P-value | 0.091 | 0.000 | |
| | 12^{th} | Change amount (%) | 0.26 ± 4.20 | −50.27 ± 2.49 | 0.000 |
| | week | compared to 0^{th} week | | | |
| | | P-value | 0.667 | 0.000 | |
| Skin | 6^{th} | Change amount (%) | 9.28 ± 1.65 | 8.16 ± 0.96 | 0.562 |
| moisture | week | compared to 0^{th} week | | | |
| | | P-value | 0.000 | 0.000 | |
| | 12^{th} | Change amount (%) | 7.00 ± 2.46 | 15.74 ± 2.06 | 0.008 |
| | week | compared to 0^{th} week | | | |
| | | P-value | 0.012 | 0.000 | |
| pH | 6^{th} | Change amount, (%) | −3.31 ± 1.09 | −3.17 ± 1.20 | 0.929 |
| | week | compared to 0^{th} week | | | |
| | | P-value | 0.002 | 0.004 | |
| | 12^{th} | Change amount (%) | −1.80 ± 1.40 | −7.18 ± 1.18 | 0.004 |
| | week | compared to 0^{th} week | | | |
| | | P-value | 0.150 | 0.000 | |

TABLE 5

| | | | | Lactic acid | P-value |
|---|---|---|---|---|---|
| | | | | bacteria | (significance |
| | | | Placebo group | intake group | between |
| PP | | | (n = 39) | (n = 39) | groups) |
| Sebum | 6^{th} | Change amount (%) | −2.35 ± 2.13 | −23.96 ± 2.24 | 0.000 |
| (μg/cm²) | week | compared to 0^{th} week | | | |
| | | P-value | 0.092 | 0.000 | |
| | 12^{th} | Change amount (%) | 1.53 ± 4.29 | −52.53 ± 2.11 | 0.000 |
| | week | compared to 0^{th} week | | | |
| | | P-value | 0.915 | 0.000 | |
| Skin | 6^{th} | Change amount (%) | 9.59 ± 1.77 | 7.71 ± 0.96 | 0.353 |
| moisture | week | compared to 0^{th} week | | | |
| | | P-value | 0.000 | 0.000 | |
| | 12^{th} | Change amount (%) | 7.20 ± 2.63 | 14.16 ± 1.96 | 0.037 |
| | week | compared to 0^{th} week | | | |
| | | P-value | 0.017 | 0.000 | |
| pH | 6^{th} | Change amount (%) | −3.99 ± 1.05 | −2.83 ± 1.20 | 0.471 |
| | week | compared to 0^{th} week | | | |
| | | P-value | 0.0002 | 0.008 | |
| | 12^{th} | Change amount (%) | −2.13 ± 1.45 | −6.87 ± 1.25 | 0.016 |
| | week | compared to 0^{th} week | | | |
| | | P-value | 0.078 | 0.000 | |

Furthermore, the skin acidity, which is one of the key indicators of skin health as described above, can be determined by the total content of lactate, a free amino acid (FAA), and the like produced from the epidermis. Accordingly, changes in the contents of the aforementioned components were also measured.

At the 0^{th}, 6^{th}, and 12^{th} weeks, epidermal tissue was collected from the foreheads and T-zone areas of the subjects using tape strips (22-mm D-SQUAME Tape, Cu Derm, USA). In the case of the lactate, a L-lactate component in an extract extracted by adding distilled water to the epidermal tissue to perform sonication was measured at 565 nm using a kit (EnzyChrom™ L-lactate assay kit, Bioassay system, USA) according to the instructions of the kit. In the case of the free amino acid, an extract extracted by performing sonication with a chloroform/methanol (2:1, v/v) solution on the tapes was measured using a kit (L-amino acid quantitation colorimetric/fluorometric kit, Biovision Co., USA) according to the instructions of the kit.

Regarding the change in the lactate content and the change in the total free amino acid content in the epidermis, which were measured in the same manner as described above, the ITT analysis results thereof are shown in Table 6 below, and the PP analysis results thereof are shown in Table 7 below.

According to the results of the ITT analysis on the change in the lactate content in the epidermis, there was no significance between groups in the placebo group and the lactic acid bacteria intake group at both the 6^{th} week and the 12^{th} week. However, in the PP analysis results, at the 12^{th} week, the lactate content of the lactic acid bacteria intake group was significantly increased, whereas the lactate content 41 the placebo group was decreased, and thus a significance between groups appeared. In the case of the total free amino acid in the epidermis, in both the ITT and PP analysis results, no significant differences between the groups were found at both the 6^{th} week and the 12^{th} week.

From the results of the PP analysis on the change in the acidity and the change in the content of the indicator component related to the acidity as described above, it was confirmed that the effect of improving the acidity by the intake of the Lactobacillus plantarum CJLP55 was due to an increase in the content of lactate.

TABLE 6

| | | | | Lactic acid | P-value |
|---|---|---|---|---|---|
| | | | | bacteria | (significance |
| | | | Placebo group | intake group | between |
| ITT | | | (n = 42) | (n = 42) | groups) |
| Lactate | 6^{th} | Change amount (%) | 37.03 ± 16.89 | 7.59 ± 14.23 | 0.186 |
| (nmol/μg | week | compared to 0^{th} week | | | |
| protein) | | P-value | 0.034 | 0.597 | |
| | 12^{th} | Change amount (%) | 3.88 ± 11.73 | 10.32 ± 13.86 | 0.724 |
| | week | compared to 0^{th} week | | | |
| | | P-value | 0.743 | 0.461 | |
| Total | 6^{th} | Change amount (%) | 47.85 ± 16.67 | 83.96 ± 30.03 | 0.296 |
| free | week | compared to 0^{th} week | | | |
| amino | | P-value | 0.006 | 0.008 | |
| acid | 12^{th} | Change amount (%) | 32.24 ± 16.40 | 45.9 ± 16.38 | 0.557 |
| (nmol/μg | week | compared to 0^{th} week | | | |
| protein) | | P-value | 0.056 | 0.008 | |

TABLE 7

| | | | | Lactic acid | P-value |
|---|---|---|---|---|---|
| | | | | bacteria | (significance |
| | | | Placebo group | intake group | between |
| PP | | | (n = 39) | (n = 39) | groups) |
| Lactate | 6^{th} | Change amount (%) | 22.47 ± 15.66 | 12.64 ± 16.25 | 0.665 |
| (nmol/μg | week | compared to 0^{th} week | | | |
| protein) | | P-value | 0.159 | 0.441 | |
| | 12^{th} | Change amount (%) | −9.76 ± 9.70 | 25.56 ± 13.65 | 0.038 |
| | week | compared to 0^{th} week | | | |
| | | P-value | 0.320 | 0.069 | |
| Total | 6^{th} | Change amount (%) | 50.81 ± 17.76 | 94.66 ± 32.43 | 0.239 |
| free | week | compared to week | | | |
| amino | | P-value | 0.007 | 0.006 | |
| acid | 12^{th} | Change amount (%) | 30.79 ± 16.98 | 51.5 ± 17.65 | 0.400 |
| (nmol/μg | week | compared to 0^{th} week | | | |
| protein) | | P-value | 0.078 | 0.006 | |

### [2-5] Checking of Change in Skin Lipid Content According to Intake of Lactic Acid Bacteria

The placebo group and the lactic acid bacteria intake group were asked to take the food, which was produced according to Experimental Example 2-2, according to the aforementioned intake method for 12 weeks, then lipid components in skin were checked, and the contents thereof were checked. At the 0^{th}, 6^{th}, and 12^{th} weeks, tapes (22-mm D-SQUAME Tape, Cu Derm, USA) were attached to the facial skin areas of the subjects, and lipids of sebum were collected. The collected tapes were immersed in chloroform/methanol (2:1, v/v) and subjected to sonication for 2 hours to extract lipids. The lipids were dried with nitrogen and dissolved in a chloroform solution again to separate triglyceride (TG), cholesterol esters (CE), free fatty acids (FFA), cholesterol (Chol), and ceramide (Cer) through HPTLC (development conditions: CHCl₃/methanol/water (10:2.5:0.25, v/v/v) as a first developing solution up by to 2 cm, CHCl₃/methanol/acetic acid (11.25:1.25:0.13, v/v/v) as a second developing solution by up to 5 cm, hexane/diethyl ether/acetone (2.5:10:0.63, v/v/v) as a third developing solution by up to 6 cm, and hexane/diethyl ether (12:0.73) as a fourth developing solution by of up to 9.5 cm were developed in this order). Respective separated fractions were scanned with a TLC III scanner and quantitatively calculated by an external standard method, and the ITT and PP analysis results thereof are shown in Tables 8 and 9 below, respectively.

Consequently, in the case of the total lipid content, as both the ITT and PP analysis results, the effect of reducing the lipid content in the lactic acid bacteria intake group showed a significance between groups from the 6^{th} week. Among them, in the case of the triglyceride, as both the ITT and PP analysis results, a significance between groups appeared with respect to the triglyceride reduction in the lactic acid bacteria intake group after the 6^{th} week, and the effect was more remarkable at the 12^{th} week. In the case of the cholesterol ester content, the reduction effect in the lactic acid bacteria intake group showed a significance between groups at the 12^{th} week. A significance between groups did not appear in the total free fatty acid content, and the reduction of the cholesterol content in the lactic acid bacteria intake group showed a significance between groups after the 6^{th} week and was more remarkable at the 12^{th} week.

In the case of the ceramide, the ceramide was fractionated into ceramides 1 to 7, and a significant between groups did not appear in the ITT analysis result. However, as the PP analysis result, the content of the ceramide 2 (Cer2) as the main ceramide was significantly increased in the lactic acid bacteria intake group at the 12^{th} week.

TABLE 8

| | | | | Lactic acid | P-value |
|---|---|---|---|---|---|
| | | | | bacteria | (significance |
| | | | Placebo group | intake group | between |
| ITT | | | (n = 42) | (n = 42) | groups) |
| Triglyceride | 6^{th} | Change amount (%) | −0.54 ± 3.93 | −19.92 ± 5.85 | 0.007 |
| (TG) | week | compared to 0^{th} week | | | |
| (nmol/μg | | P-value | 0.112 | 0.0001 | |
| protein) | 12^{th} | Change amount (%) | 4.56 ± 5.76 | −27.28 ± 4.65 | 0.000 |
| | week | compared to 0^{th}week | | | |
| | | P-value | 0.784 | 0.000 | |
| Cholesterol | 6^{th} | Change amount (%) | −6.77 ± 3.76 | −19.68 ± 8.84 | 0.183 |
| esters (CE) | week | compared to 0^{th} week | | | |
| (nmol/μg | | P-value | 0.004 | 0.000 | |
| protein) | 12^{th} | Change amount (%) | 2.89 ± 4.92 | −22.66 ± 8.93 | 0.014 |
| | week | compared to 0^{th} week | | | |
| | | P-value | 0.531 | 0.000 | |
| Free fatty | 6^{th} | Change amount (%) | −4.13 ± 5.22 | −19.17 ± 5.93 | 0.060 |
| acid (FFA) | week | compared to 0^{th} week | | | |
| (nmol/μg | | P-value | 0.020 | 0.001 | |
| protein) | 12^{th} | Change amount (%) | 10.80 ± 7.81 | −9.31 ± 7.73 | 0.071 |
| | week | compared to 0^{th} week | | | |
| | | P-value | 0.682 | 0.006 | |
| Cholesterol | 6^{th} | Change amount (%) | −2.27 ± 6.73 | −29.69 ± 5.27 | 0.002 |
| (Chol) | week | compared to 0^{th} week | | | |
| (nmol/μg | | P-value | 0.217 | 0.000 | |
| protein) | 12^{th} | Change amount (%) | 5.25 ± 12.69 | −40.08 ± 3.89 | 0.001 |
| | week | compared to 0^{th} week | | | |
| | | P-value | 0.746 | 0.000 | |
| Ceramide 2 | 6^{th} | Change amount (%) | 132.97 ± 68.00 | 185.43 ± 90.83 | 0.645 |
| (Cer2) | week | compared to 0^{th} week | | | |
| (nmol/μg | | P-value | 0.057 | 0.048 | |
| protein) | 12^{th} | Change amount (%) | 17.17 ± 8.36 | 22.27 ± 7.41 | 0.649 |
| | week | compared to 0^{th} week | | | |
| | | P-value | 0.047 | 0.005 | |
| Ceramides 1 | 6^{th} | Change amount (%) | 100.27 ± 73.43 | 129.12 ± 50.92 | 0.748 |
| and 3 to 7 | week | compared to 0^{th}week | | | |
| (nmol/μg | | P-value | 0.180 | 0.015 | |
| protein) | 12^{th} | Change amount (%) | 8.09 ± 10.52 | −2.12 ± 10.45 | 0.493 |
| | week | compared to 0^{th} week | | | |
| | | P-value | 0.446 | 0.840 | |
| Total lipid | 6^{th} | Change amount (%) | −4.98 ± 3.32 | −22.63 ± 5.08 | 0.005 |
| (nmol/μg | week | compared to 0^{th} week | | | |
| protein) | | P-value | 0.009 | 0.000 | |
| | 12^{th} | Change amount (%) | 2.61 ± 4.53 | −26.67 ± 4.38 | 0.000 |
| | week | compared to 0^{th} week | | | |
| | | P-value | 0.616 | 0.000 | |

TABLE 9

| | | | | Lactic acid | P-value |
|---|---|---|---|---|---|
| | | | | bacteria | (significance |
| | | | Placebo group | intake group | between |
| PP | | | (n = 39) | (n = 39) | groups) |
| Triglyceride | 6^{th} | Change amount (%) | −0.73 ± 4.22 | −21.13 ± 6.22 | 0.008 |
| (TG) | week | compared to 0^{th} week | | | |
| (nmol/μg | | P-value | 0.112 | 0.0001 | |
| protein) | 12^{th} | Change amount (%) | 4.05 ± 6.19 | −27.18 ± 4.95 | 0.0002 |
| | week | compared to 0^{th} week | | | |
| | | P-value | 0.720 | 0.000 | |
| Cholesterol | 6^{th} | Change amount (%) | −7.16 ± 4.02 | −19.95 ± 9.48 | 0.218 |
| esters (CE) | week | compared to 0^{th} week | | | |
| (nmol/μg | | P-value | 0.006 | 0.000 | |
| protein) | 12^{th} | Change amount (%) | 2.79 ± 5.27 | −21.72 ± 9.59 | 0.028 |
| | week | compared to 0^{th} week | | | |
| | | P-value | 0.555 | 0.000 | |
| Free fatty | 6^{th} | Change amount (%) | −3.64 ± 5.60 | −17.63 ± 6.32 | 0.100 |
| acid (FFA) | week | compared to 0^{th} week | | | |
| (nmol/μg | | P-value | 0.026 | 0.003 | |
| protein) | 12^{th} | Change amount (%) | 8.91 ± 7.85 | −6.99 ± 8.20 | 0.165 |
| | week | compared to 0^{th} week | | | |
| | | P-value | 0.658 | 0.019 | |
| Cholesterol | 6^{th} | Change amount. (%) | −2.63 ± 7.18 | −29.71 ± 5.60 | 0.004 |
| (Chol) | week | compared to 0^{th} week | | | |
| (nmol/μg | | P-value | 0.227 | 0.0001 | |
| protein) | 12^{th} | Change amount (%) | 6.12 ± 13.65 | −38.83 ± 4.12 | 0.003 |
| | week | compared to 0^{th}week | | | |
| | | P-value | 0.751 | 0.000 | |
| Ceramide 2 | 6^{th} | Change amount (%) | 116.77 ± 32.68 | 116.77 ± 32.68 | 0.855 |
| (Cer2) | week | compared to 0^{th} week | | | |
| (nmol/μg | | P-value | 0.001 | 0.001 | |
| protein) | 12^{th} | Change amount (%) | 29.94 ± 7.33 | 29.94 ± 7.33 | 0.020 |
| | week | compared to 0^{th} week | | | |
| | | P-value | 0.000 | 0.000 | |
| Ceramides 1 | 6^{th} | Change amount (%) | 97.32 ± 39.20 | 97.32 ± 39.20 | 0.928 |
| and 3 to 7 | week | compared to 0^{th} week | | | |
| (nmol/μg | | P-value | 0.018 | 0.018 | |
| protein) | 12^{th} | Change amount (%) | −3.70 ± 11.18 | −3.70 ± 11.18 | 0.766 |
| | week | compared to 0^{th} week | | | |
| | | P-value | 0.742 | 0.742 | |
| Total lipid | 6^{th} | Change amount (%) | −5.27 ± 3.55 | −23.03 ± 5.42 | 0.008 |
| (nmol/μg | week | compared to 0^{th} week | | | |
| protein) | | P-value | 0.011 | 0.000 | |
| | 12^{th} | Change amount (%) | 2.21 ± 4.84 | −25.90 ± 4.67 | 0.0001 |
| | week | compared to 0^{th} week | | | |
| | | P-value | 0.593 | 0.000 | |

When the results of the human application test as described above are combined, the intake of the maltodextrin and powdered glucose contained as excipients of the food taken by the test subjects suppresses the secretion of some lipids from epidermal cells and sebaceous glands only at the 6^{th} week of the intake, whereas the intake of the Lactobacillus plantarum CJLP55 according to the present application causes an effect of reducing the total content of sebum by suppressing the content and secretion of lipids such as triglyceride, cholesterol, and cholesterol esters, and has an effect of maintaining skin moisturizing by selectively increasing the production of ceramides from epidermal cells, and thus it is expected that an effect that can improve the severity of acne ultimately occurs.

### [2-6] Checking of Effect of Reducing Harmful Skin Bacteria According to Intake of Lactic Acid Bacteria

In order to analyze the change in the flora of harmful skin bacteria proliferating in skin according to the intake of the Lactobacillus plantarum CJLP55, first, a sample of the skin surface was collected by performing rubbing on the same area of one cheek for 30 seconds and stored at −80° C. Each skin sample obtained at the 0^{th} week and the 12^{th} week was subjected to next generation sequencing (NGS, MiSeq) for the 16S rRNA V3-V4 region by requesting Macrogen, Inc., and the change in the skin flora was analyzed through pre-processing and data analysis of base sequence data obtained as described above.

Since DNA libraries could not be produced from samples from one subject in the placebo group and three subjects in the lactic acid bacteria intake group among the test subjects, the ITT analysis targeted 41 subjects in the placebo group and 39 subjects in the lactic acid bacteria intake group, the PP analysis targeted 38 subjects in the placebo group and 36 subjects in the lactic acid bacteria intake group, and the results thereof are shown in Table 10below. As both the ITT and PP analysis results, it was confirmed that when the lactic acid bacteria intake group was compared to the placebo group, there was a significant difference in the proportion of microorganisms belonging to the genus Propionibacterium or the genus Staphylococcus at the 12^{th} week. In the case of the placebo group, the microorganisms belonging to the genus Propionibacterium or the genus Staphylococcus and known as harmful skin bacteria were significantly increased compared to the 0^{th} week, whereas in the case of the lactic acid bacteria intake group, there was no significant change for 12 weeks, and thus it could be confirmed that the proliferation of the harmful bacteria was suppressed.

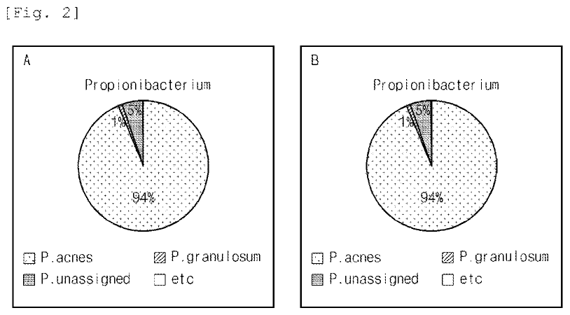

Furthermore, as a result of performing species-level analysis on the sequences assigned to the genus Propionibacterium, it was confirmed that 94% of the sequence on average was P. acnes in both the ITT and PP analysis (FIG. 2).

TABLE 10

| | | | | P-value |
|---|---|---|---|---|
| | | | Lactic acid | (significance |
| | | | bacteria | between |
| | Bacteria | Placebo group | intake group | groups) |
| | | (n = 41) | (n = 39) | |
| ITT | Genus Propionibacterium | 15.98% | 9.51% | 0.0154 |
| analysis | Genus Staphylococcus | 6.29% | 4.30% | 0.0376 |
| | | (n = 38) | (n = 36) | |
| PP | Genus Propionibacterium | 15.44% | 9.07% | 0.0080 |
| analysis | Genus Staphylococcus | 6.66% | 4.59% | 0.0364 |

### [2-7] Checking of Change in Skin Roughness According to Intake of Lactic Acid Bacteria

The placebo group and the lactic acid bacteria intake group were asked to take the food, which was produced according to Experimental Example 2-2, according to the aforementioned intake method for 12 weeks, and then the change in the skin roughness was checked by measuring the amount of the removed keratin. At the 0^{th}, 6^{th}, and 12^{th} weeks, skin keratin layers were collected using tape strips from the volar forearm of the subjects, photographs were taken at a fixed position of a densitometer (SLB Myimager, Seoulin, Korea), and the amounts of the removed keratin were measured with the densitometer, digitized, and compared. The amount of keratin for each week of each subject was calculated as the average of the measured values for five tape strips, the ITT analysis results thereof are shown in Table 11 below, and the PP analysis results thereof are shown in Table 12 below.

In the case of the results of the ITT analysis on the skin roughness measured through digitization of the keratin of each group, there was no significance between groups with respect to the changes at the 6^{th} week and the 12^{th} week, but in the case of the PP analysis, it was confirmed that the skin roughness of the lactic acid bacteria intake group was significantly reduced compared to the placebo group, a significance between groups appeared with respect to the roughness improvement of the lactic acid bacteria intake group from the 6^{th} week, and the reduction effect was more remarkable at the 12^{th} week.

As seen from the above results, in the subjects who took the Lactobacillus plantarum CJLP55, the keratin was reduced and the skin roughness was reduced, which means that the effect of maintaining skin moisturizing was improved by the intake of the aforementioned lactic acid bacteria.

TABLE 11

| | | | | Lactic acid | P-value |
|---|---|---|---|---|---|
| | | | | bacteria | (significance |
| | | | Placebo group | intake group | between |
| ITT | | | (n = 42) | (n = 42) | groups) |
| Skin | 6^{th} | Change amount | −3.92 ± 2.33 | −8.10 ± 3.14 | 0.288 |
| roughness | week | (%) compared to | | | |
| (mean raw | | 0^{th} week | | | |
| density) | | P-value | 0.100 | 0.014 | |
| | 12^{th} | Change amount | −8.81 ± 2.27 | −12.20 ± 2.76 | 0.346 |
| | week | (%) compared to | | | |
| | | 0^{th} week | | | |
| | | P-value | 0.0001 | 0.0001 | |

TABLE 12

| | | | | Lactic acid | P-value |
|---|---|---|---|---|---|
| | | | | bacteria | (significance |
| | | | Placebo group | intake group | between |
| PP | | | (n = 39) | (n = 39) | groups) |
| Skin | 6^{th} | Change amount | −2.87 ± 2.38 | −10.58 ± 3.02 | 0.048 |
| roughness | week | (%) compared to | | | |
| (mean raw | | 0^{th} week | | | |
| density) | | P-value | 0.183 | 0.001 | |
| | 12^{th} | Change amount | −6.72 ± 2.07 | −14.31 ± 2.68 | 0.028 |
| | week | (%) compared to | | | |
| | | 0^{th} week | | | |
| | | P-value | 0.0001 | 0.0001 | |

Hereinbefore, representative experimental examples of the present application have been exemplarily described, but the scope of the present application is not limited to only the specific experimental examples described above, and could be appropriately modified by a person with ordinary skill in the art within the scope described in the claims of the present application.

1-10. (canceled)

11. A method for improving skin conditions in a subject comprising administering to the subject an effective amount of a microorganism deposited under the accession number KCTC 11401BP and/or a culture solution thereof.

12. The method of claim 11, wherein a composition containing the microorganism and/or the culture solution thereof in an amount of 3 wt % to 15 wt % is administered to the subject.

13. The method of claim 11, wherein the method further comprises administering maltodextrin and glucose to the subject.

14. The method of claim 11, wherein the administering comprises an oral administration.

15. The method of claim 11, wherein the improving skin conditions comprises one or more selected from the group consisting of sebum suppression, sebum component improvement, acne improvement, and dermatitis improvement.

16. The method of claim 11, wherein the method reduces one or more selected from the group consisting of a triglyceride, cholesterol, a cholesterol ester, and a free fatty acid in sebum, or increases a ceramide in the subject.

17. The method of claim 11, wherein the method reduces oil in skin and increases moisture in skin in the subject.

18. The method of claim 11, wherein the method improves an acidity of skin in the subject.

19. The method of claim 11, wherein the method increases antibacterial activity against microorganisms belonging to the genus Propionibacterium or the genus Staphylococcus in the subject.

20. The method of claim 19, wherein the microorganisms belonging to the genus Propionibacterium are Propionibacterium acnes.
